SEQUENCE LISTING (5 sequences)

SEQ ID NO: 1 - protein, 976 aa, Homo sapiens [sequence omitted]

SEQ ID NO: 2 - DNA, 30 bp, Homo sapiens
  Primer
aagaagagatggtacctcgaggggtgaccc

SEQ ID NO: 3 - DNA, 33 bp, Homo sapiens
  Primer
ctgcttcgcggccgcgttaactcttctcaacca

SEQ ID NO: 4 - DNA, 20 bp, Homo sapiens
  Primer
agctcgtttagtgaaccgtc

SEQ ID NO: 5 - DNA, 20 bp, Homo sapiens
  Primer
gtcagacaaaatgatgcaac

(19) United States
(12) Patent Application Publication
(10) Pub. No.: US 2004/0241226 A1
(43) Pub. Date: Dec. 2, 2004

(54) Use of potent, selective and non-toxic c-kit inhibitors for treating bacterial infections

(76) Inventors: Alain Moussy, Paris (FR); Jean-Pierre Kinet, Lexington, MA (US)

Correspondence Address: SUGHRUE MION, PLLC, 2100 PENNSYLVANIA AVENUE, N.W., SUITE 800, WASHINGTON, DC, 20037, US

(21) Appl. No.: 10/490,287

(22) PCT Filed: Sep. 20, 2002
(86) PCT No.: PCT/IB02/04251

Related U.S. Application Data
(60) Provisional application No. 60/323,313, filed on Sep. 20, 2001.

(51) Int. Cl. A61K 31/496; A61K 31/519; A61K 9/20

(52) U.S. Cl. 424/465; 514/249; 514/253.08; 514/265.1; 514/312

(57) ABSTRACT

The present invention relates to a method for treating bacterial infections, preferably infections caused by FimH expressing bacteria, comprising administering a tyrosine kinase inhibitor to a human in need of such treatment, more particularly a non toxic, potent and selective c-kit inhibitor, wherein said inhibitor is unable to promote detah of IL-3 dependent cells cultured to presence of IL-3.

The present invention relates to a method for treating bacterial infections, preferably infections caused by FimH expressing bacteria, comprising administering a tyrosine kinase inhibitor to a human in need of such treatment, more particularly a non toxic, potent and selective c-kit inhibitor, wherein said inhibitor is unable to promote death of IL-3 dependent cells cultured in presence of IL-3.

Bacterial infections are the most common diseases among mammalian and yet they remain deadly in case of resistant strains appearance. Resistance primarily originates from the extensive use of antibiotics. Antibiotics are agents acting on the bacterial cell wall such as bacitracin, the cephalosporins, and the penicillins, agents capable of inhibiting replication and protein synthesis by their effects on ribosomes, such as the aminoglycosides, the tetracyclines, the streptomycins and the macrolide antibiotics such as erythromycin; agents affecting nucleic acid metabolism, such as the fluoroquinolones, actinomycin; and drugs affecting intermediary metabolism, such as the sulfonamides and trimethoprim.

Despite the efficacy of antibiotics, few bacteria occasionally acquire mutations under high selection pressure, which renders the above mentioned antibiotic molecular targets insensitive and leads to the birth of new resistant strains.

More recently, muti-resistant strains have been observed during nosocomial infections and has come to the attention of the public. Facing the emergence of these deadly strains, research has focused on other mechanisms leading to multi-resistance. For example, it has been found that the marA loci confers multiple antibiotic resistance via increased efflux of many structurally unrelated antibiotics (McMurry et al., Antimicrob. Agents Chemother. 38:542-546, 1994). Multi-drug efflux pumps are now generally thought to be responsible for drugs insensitivity.

However, this mechanism leading to the resistance of bacteria does not explain the recurrence observed in bacterial infections. Indeed, after eradication of the bacteria, resurgence is observed later on suggesting that a small portion of bacteria were able to survive and remain concealed in the body. For example, urinary tract infections (UTI) have been treated for years with the antibiotics Bactrim, Macrodantin and a combination of Sulfa drugs that offer quick relief, but these antibiotics become useless after several prescriptions because the infection looks as if it has settled in the body and re-emerges from time to time.

Therefore, there is a need for new medications that would prevent and treat resurgence of bacterial infections.

In connection with the invention, it is postulated that bacteria, especially FimH expressing bacteria, are capable of escaping the immune system as well as the action of antibiotics by integration into mast cells, in which they remain concealed for a period time.

Mast cells (MC) are tissue elements derived from a particular subset of hematopoietic stem cells that express CD34, c-kit and CD13 antigens (Kirshenbaum et al, Blood. 94: 2333-2342, 1999 and Ishizaka et al, Curr Opin Immunol. 5: 937-43, 1993). Immature MC progenitors circulate in the bloodstream and differentiate in tissues. These differentiation and proliferation processes are under the influence of cytokines, one of utmost importance being Stem Cell Factor (SCF), also termed Kit ligand (KL), Steel factor (SL) or Mast Cell Growth Factor (MCGF). SCF receptor is encoded by the protooncogene c-kit, that belongs to type III receptor tyrosine kinase subfamily (Boissan and Arock, J Leukoc Biol. 67: 135-48, 2000). This receptor is also expressed on others hematopoietic or non hematopoietic cells. Ligation of c-kit receptor by SCF induces its dimerization followed by its transphosphorylation, leading to the recruitement and activation of various intracytoplasmic substrates. These activated substrates induce multiple intracellular signaling pathways responsible for cell proliferation and activation (Boissan and Arock, 2000). Mast cells are characterized by their heterogeneity, not only regarding tissue location and structure but also at the functional and histochemical levels (Aldenborg and Enerback., Histochem. J. 26: 587-96, 1994; Bradding et al. J Immunol. 155: 297-307, 1995; Irani et al, J Immunol. 147: 247-53, 1991; Miller et al, Curr Opin Immunol. 1: 637-42, 1989 and Welle et al, J Leukoc Biol. 61: 233-45, 1997).

Apart from their key role as effector cells of allergic and potentially lethal anaphylactic reactions, mast cells might contribute to the initiation of acquired immune reactions. Indeed, mast cells can phagocytosize diverse particles, and particularly bacteria. For example, recent studies have implicated rodent mast cells in the innate immune response to infectious bacteria and have shown that human mast cells are intrinsically capable of mediating microbial recognition and of actively contributing to the host defense against bacteria; Arock M et al, Infect Immun 1998 December;66(12):6030-4.

Galli S J et al, Curr Opin Immunol 1999 February;11(1):53-9 suggested that mast cell function can be manipulated for therapeutic ends using SCF to boost immune response. This was also proposed by Maurer et al, J Exp Med 1998 Dec. 21;188(12):2343-8 who identified c-kit and mast cells as potential therapeutic targets for enhancing innate immune responses.

While this can be acknowledged as far as acute infections are concerned, it could have serious drawbacks when considering recurrent bacterial infections.

Indeed, mast cells display very peculiar cell membrane structures called caveolae. Caveolae are subcellular structures implicated in the import and transcytosis of macromolecules and in transmembrane signaling. The composition and function of caveolae is reviewed in Anderson R G, Annu Rev Biochem 1998;67:199-225. In this article, caveolae are presented not just as an endocytic device with a peculiar membrane shape but rather as an entire membrane system with multiple functions essential for the cell. It is also mentioned that pathogens have been identified that use it as a means of gaining entrance to the cell.

Shin J S et al, Science. 2000 Aug. 4;289(5480):732-3 reported that caveolae were detected in the microvilli and intracellular vesicles of cultured mouse bone marrow-derived mast cells (BMMCs). CD48, a receptor for FimH-expressing (type 1 fimbriated) Escherichia coli, was specifically localized to plasmalemmal caveolae in BMMCs. The involvement of caveolae in bacterial entry into BMMCs was demonstrated because caveolae-disrupting and -usurping agents specifically blocked E. coli entry. More importantly, it was demonstrated that some microbes utilize the unique features of caveolae to enter and traffic, without any apparent loss of viability and function, to different sites within immune and other host cells; Shin & Abraham, Immunology 2001, 102 (1), 2-7.

Therefore, bacteria-encapsulating caveolar chambers in mast cells form a reservoir of surviving bacteria that is postulated here to be implicated in the resurgence of infections.

FimH, a mannose-binding lectin, expressed by many enterobacteria including E. coli, K. pneumoniae and S. typhimurium, binds to the receptor CD48 present at the surface of caveolae, Shin J S et al, FEMS Microbiol Lett 2001 Apr. 13;197(2):131-8. As a result, FimH expressing bacteria enter inside mast cells and remain concealed and viable in caveolar chambers. In addition, Abraham S N et al, Nature 1988 Dec. 15;336(6200):682-4 have observed a conservation of the D-mannose-adhesion protein among type 1 fimbriated members of the family Enterobacteriaceae.

It is proposed here that at some point, exocytosis of these chambers leads to the release of intact and living bacteria, which are responsible for the resurgence of the infection.

Consequently, apart from being beneficial for the organism through its ability to initiate immune responses towards a variety of pathogens, the mast cell may also be detrimental for the host during recurrent infectious diseases as specified above.

In such detrimental circumstances, therapeutic strategies aiming at blocking the activation and the survival of mast cells, for instance through inhibition of c-kit or c-kit signaling is proposed to decrease the inappropriate release of inflammatory mediators, as well as the survival of intracellular pathogens.

Therefore, the invention provides a new therapeutic strategy aimed at the use of c-kit specific kinase inhibitors to inhibit mast cell proliferation, survival and activation. A new route for treating recurrent bacterial infections is provided, which consists of destroying mast cells that constitute a reservoir for bacteria. It has been found that tyrosine kinase inhibitors and more particularly c-kit inhibitors are especially suited to reach this goal.

### DESCRIPTION

The present invention relates to a method for treating bacterial infections comprising administering a tyrosine kinase inhibitor to a mammalian in need of such treatment, wherein said inhibitor is unable to promote death of IL-3 dependent cells cultured in presence of IL-3.

Tyrosine kinase inhibitors are selected for example from bis monocyclic, bicyclic or heterocyclic aryl compounds (WO 92/20642), vinylene-azaindole derivatives (WO 94/14808) and 1-cycloproppyl-4-pyridyl-quinolones (U.S. Pat. No. 5,330,992), Styryl compounds (U.S. Pat. No. 5,217,999), styryl-substituted pyridyl compounds (U.S. Pat. No. 5,302,606), seleoindoles and selenides (WO 94/03427), tricyclic polyhydroxylic compounds (WO 92/21660) and benzylphosphonic acid compounds (WO 91/15495), pyrimidine derivatives (U.S. Pat. No. 5,521,184 and WO 99/03854), indolinone derivatives and pyrrol-substituted indolinones (U.S. Pat. No. 5,792,783, EP 934 931, U.S. Pat. No. 5,834,504, U.S. Pat. No. 5,883,116, U.S. Pat. No. 5,883,113, U.S. Pat. No. 5,886,020, WO 96/40116 and WO 00/38519), as well as bis monocyclic, bicyclic aryl and heteroaryl compounds (EP 584 222, U.S. Pat. No. 5,656,643 and WO 92/20642), quinazoline derivatives (EP 602 851, EP 520 722, U.S. Pat. No. 3,772,295 and U.S. Pat. No. 4,343,940) and aryl and heteroaryl quinazoline (U.S. Pat. No. 5,721,237, U.S. Pat. No. 5,714,493, U.S. Pat. No. 5,710,158 and WO 95/15758).

Preferably, said tyrosine kinase inhibitors are non-toxic, selective and potent c-kit inhibitors. Such inhibitors can be selected from the group consisting of indolinones, pyrimidine derivatives, pyrrolopyrimidine derivatives, quinazoline derivatives, quinoxaline derivatives, pyrazoles derivatives, bis monocyclic, bicyclic or heterocyclic aryl compounds, vinylene-azaindole derivatives and pyridyl-quinolones derivatives, styryl compounds, styryl-substituted pyridyl compounds, , seleoindoles, selenides, tricyclic polyhydroxylic compounds and benzylphosphonic acid compounds.

Among preferred compounds, it is of interest to focus on pyrimidine derivatives such as N-phenyl-2-pyrimidine-amine derivatives (U.S. Pat. No. 5,521,184 and WO 99/03854), indolinone derivatives and pyrrol-substituted indolinones (U.S. Pat. No. 5,792,783, EP 934 931, U.S. Pat. No. 5,834,504), U.S. Pat. No. 5,883,116, U.S. Pat. No. 5,883,113, U.S. Pat. No. 5,886,020, WO 96/40116 and WO 00/38519), as well as bis monocyclic, bicyclic aryl and heteroaryl compounds (EP 584 222, U.S. Pat. No. 5,656,643 and WO 92/20642), quinazoline derivatives (EP 602 851, EP 520 722, U.S. Pat. No. 3,772,295 and U.S. Pat. No. 4,343,940), 4-amino-substituted quinazolines (U.S. Pat. No. 3,470,182), 4-thienyl-2-(1H)-quinazolones, 6,7-dialkoxyquinazolines (U.S. Pat. No. 3,800,039), aryl and heteroaryl quinazoline (U.S. Pat. No. 5,721,237, U.S. Pat. No. 5,714,493, U.S. Pat. No. 5,710,158 and WO 95/15758), 4-anilinoquinazoline compounds (U.S. Pat. No. 4,464,375), and 4-thienyl-2-(1H)-quinazolones (U.S. Pat. No. 3,551,427).

So, preferably, the invention relates to a method for treating bacterial infections comprising administering a non toxic, potent and selective c-kit inhibitor which is a pyrimidine derivative, more particularly N-phenyl-2-pyrimidine-amine derivatives of formula I:

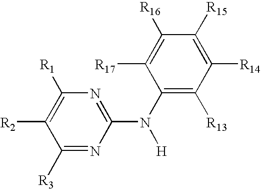

wherein the R1, R2, R3, R13 to R17 groups have the meanings depicted in EP 564 409 B1, incorporated herein in the description.

Preferably, the N-phenyl-2-pyrimidine-amine derivative is selected from the compounds corresponding to formula II:

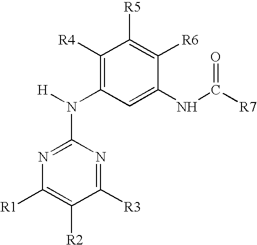

Wherein R1, R2 and R3 are independently chosen from H, F, Cl, Br, I, a C1-C5 alkyl or a cyclic or heterocyclic group, especially a pyridyl group;

R4, R5 and R6 are independently chosen from H, F, Cl, Br, I, a C1-C5 alkyl, especially a methyl group;

and R7 is a phenyl group bearing at least one substituent, which in turn possesses at least one basic site, such as an amino function.

Preferably, R7 is the following group:

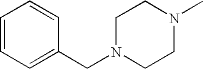

Among these compounds, the preferred are defined as follows:

R1 is a heterocyclic group, especially a pyridyl group,

R2 and R3 are H,

R4 is a C1-C3 alkyl, especially a methyl group,

R5 and R6 are H,

and R7 is a phenyl group bearing at least one substituent, which in turn possesses at least one basic site, such as an amino function, for example the group:

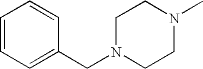

Therefore, in a preferred embodiment, the invention relates to a method for treating bacterial infections comprising the administration of an effective amount of the compound known in the art as CGP57148B:

4-(4-méhylpiperazine-1-ylméthyl)-N-[4-méthyl-3-(4-pyridine-3-yl)pyrimidine-2 ylamino)phényl]-benzamide corresponding to the following formula:

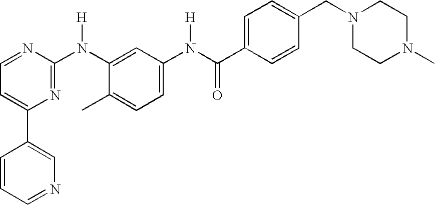

The preparation of this compound is described in example 21 of EP 564 409 and the β-form, which is particularly useful is described in WO 99/03854.

Alternatively, the c-kit inhibitor can be selected from:

indolinone derivatives, more particularly pyrrol-substituted indolinones,

monocyclic, bicyclic aryl and heteroaryl compounds, quinazoline derivatives,

and quinaxolines, such as 2-phényl-quinaxoline derivatives, for example 2-phenyl-6,7-dimethoxy quinaxoline.

In a preferred aspect, the invention contemplates the method mentioned above, wherein said c-kit inhibitor is unable to promote death of IL-3 dependent cells cultured in presence of IL-3.

The expression “bacterial infections” will be understood herein as recurrent bacterial infections, more particularly resurging infections after asymptomatic periods. Preferably, bacteria are FimH expressing bacteria such as Gram-negative enterobacteria which include but are not limited to well known pathogenic species such as E. coli, Klebsiella pneumoniae, Serratia marcescens, Citrobactor freudii and Salmonella typhimurium. In connection with the invention, bacterial infections encompass recurrent urinary tract infections such as bacterial cystitis and respiratory tract infections.

In another embodiment, c-kit inhibitors as mentioned above are inhibitors of activated c-kit. In frame with the invention, the expression “activated c-kit” means a constitutively activated-mutant c-kit including at least one mutation selected from point mutations, deletions, insertions, but also modifications and alterations of the natural c-kit sequence (SEQ ID No 1). Such mutations, deletions, insertions, modifications and alterations can occur in the transphosphorylase domain, in the juxtamembrane domain as well as in any domain directly or indirectly responsible for c-kit activity. The expression “activated c-kit” also means herein SCF-activated c-kit. Preferred and optimal SCF concentrations for activating c-kit are comprised between 5.10⁻⁷ M and 5.10⁻⁶ M, preferably around 2.10⁻⁶ M. In a preferred embodiment, the activated-mutant c-kit in step a) has at least one mutation proximal to Y823, more particularly between amino acids 800 to 850 of SEQ ID No 1 involved in c-kit autophosphorylation, notably the D816V, D816Y, D816F and D820G mutants. In another preferred embodiment, the activated-mutant c-kit in step a) has a deletion in the juxtamembrane domain of c-kit. Such a deletion is for example between codon 573 and 579 called c-kit d(573-579). The point mutation V559G proximal to the juxtamembrane domain c-kit is also of interest.

In this regard, the invention contemplates a method for treating bacterial infections comprising administering to a mammalian in need of such treatment a compound that is a selective, potent and non toxic inhibitor of activated c-kit obtainable by a screening method which comprises:

a) bringing into contact (i) activated c-kit and (ii) at least one compound to be tested; under conditions allowing the components (i) and (ii) to form a complex,

b) selecting compounds that inhibit activated c-kit,

c) testing and selecting a subset of compounds identified in step b), which are unable to promote death of IL-3 dependent cells cultured in presence of IL-3.

This screening method can further comprise the step consisting of testing and selecting a subset of compounds identified in step b) that are inhibitors of mutant activated c-kit (for example in the transphosphorylase domain), which are also capable of inhibiting SCF-activated c-kit wild.

Alternatively, in step a) activated c-kit is SCF-activated c-kit wild.

A best mode for practicing this method consists of testing putative inhibitors at a concentration above 10 μM in step a). Relevant concentrations are for example 10, 15, 20, 25, 30, 35 or 40 μM.

In step c), IL-3 is preferably present in the culture media of IL-3 dependent cells at a concentration comprised between 0.5 and 10 ng/ml, preferably between 1 to 5 ng/ml.

Examples of IL-3 dependent cells include but are not limited to:

cell lines naturally expressing and depending on c-kit for growth and survival. Among such cells, human mast cell lines can be established using the following procedures: normal human mast cells can be infected by retroviral vectors containing sequences coding for a mutant c-kit comprising the c-kit signal peptide and a TAG sequence allowing to differentiate mutant c-kits from c-kit wild expressed in hematopoetic cells by means of antibodies.

This technique is advantageous because it does not induce cellular mortality and the genetic transfer is stable and gives satisfactory yields (around 20%). Pure normal human mast cells can be routinely obtained by culturing precursor cells originating from blood obtained from human umbilical vein. In this regard, heparinated blood from umbilical vein is centrifuged on a Ficoll gradient so as to isolate mononucleated cells from other blood components. CD34+ precursor cells are then purified from the isolated cells mentioned above using the immunomagnetic selection system MACS (Miltenyi biotech). CD34+ cells are then cultured at 37° C. in 5% CO₂ atmosphere at a concentration of 10⁵ cells per ml in the medium MCCM (α-MEM supplemented with L-glutamine, penicillin, streptomycin, 5 10⁻⁵ M β-mercaptoethanol, 20% veal fœtal serum, 1% bovine albumin serum and 100 ng/ml recombinant human SCF. The medium is changed every 5 to 7 days. The percentage of mast cells present in the culture is assessed each week, using May-Grünwal Giemsa or Toluidine blue coloration. Anti-tryptase antibodies can also be used to detect mast cells in culture. After 10 weeks of culture, a pure cellular population of mast cells (<98%) is obtained.

It is possible using standard procedures to prepare vectors expressing c-kit for transfecting the cell lines established as mentioned above. The cDNA of human c-kit has been described in Yarden et al., (1987) EMBO J.6 (11), 3341-3351. The coding part of c-kit (3000 bp) can be amplified by PCR and cloned, using the following oligonucleotides:

5′AAGAAGAGATGGTACCTCGAGGGGTGACCC3′
(SEQ ID No2) sens
5′CTGCTTCGCGGCCGCGTTAACTCTTCTCAACCA3′
(SEQ ID No3) antisens

The PCR products, digested with Not1 and Xho1, has been inserted using T4 ligase in the pFlag-CMV vector (SIGMA), which vector is digested with Not1 and Xho1 and dephosphorylated using CIP (Biolabs). The pFlag-CMV-c-kit is used to transform bacterial clone XL1-blue. The transformation of clones is verified using the following primers:

| | | |
|---|---|---|
| 5′AGCTCGTTTAGTGAACCGTC3′ | (SEQ ID No4) | sens, |
| 5′GTCAGACAAAATGATGCAAC3′ | (SEQ ID No5) | antisens. |

Directed mutagenesis is performed using relevant cassettes is performed with routine and common procedure known in the art.

The vector Migr-1 (ABC) can be used as a basis for constructing retroviral vectors used for transfecting mature mast cells. This vector is advantageous because it contains the sequence coding for GFP at the 3′ and of an IRES. These features allow to select cells infected by the retrovirus using direct analysis with a fluorocytometer. As mentioned above, the N-terminal sequence of c-kit c-DNA can be modified so as to introduce a Flag sequence that will be useful to discriminating heterogeneous from endogenous c-kit.

Other IL-3 dependent cell lines that can be used include but are not limited to:

BaF3 mouse cells expressing wild-type or mutated form of c-kit (in the juxtamembrane and in the catalytic sites) are described in Kitayama et al, (1996), Blood 88, 995-1004 and Tsujimura et al, (1999), Blood 93, 1319-1329.

IC-2 mouse cells expressing either c-kit^{WT} or c-kit^{D814Y} are presented in Piao et al, (1996), Proc. Natl. Acad. Sci. USA 93, 14665-14669.

IL-3 independent cell lines are:

HMC-1, a factor-independent cell line derived from a patient with mast cell leukemia, expresses a juxtamembrane mutant c-kit polypeptide that has constitutive kinase activity (Furitsu T et al, J Clin Invest. 1993;92:1736-1744; Butterfield et al, Establishment of an immature mast cell line from a patient with mast cell leukemia. Leuk Res. 1988;12:345-355 and Nagata et al, Proc Natl Acad Sci USA. 1995;92:10560-10564).

P815 cell line (mastocytoma naturally expressing c-kit mutation at the 814 position) has been described in Tsujimura et al, (1994), Blood 83, 2619-2626.

The extent to which component (ii) inhibits activated c-kit can be measured in vitro or in vivo. In case it is measured in vivo, cell lines expressing an activated-mutant c-kit, which has at least one mutation proximal to Y823, more particularly between amino acids 800 to 850 of SEQ ID No1 involved in c-kit autophosphorylation, notably the D816V, D816Y, D816F and D820G mutants, are preferred.

Example of cell lines expressing an activated-mutant c-kit are as mentioned above.

In another preferred embodiment, the method further comprises the step consisting of testing and selecting compounds capable of inhibiting c-kit wild at concentration below 1 μM. This can be measured in vitro or in vivo.

Therefore, compounds are identified and selected according to the method described above are potent, selective and non-toxic c-kit wild inhibitors.

Alternatively, the screening method according to the invention can be practiced in vitro In this regard, the inhibition of mutant-activated c-kit and/or c-kit wild can be measured using standard biochemical techniques such as immunoprecipitation and western blot. Preferably, the amount of c-kit phosphorylation is measured.

In a still further embodiment, the invention contemplates a method for treating bacterial infections as depicted above wherein the screening comprises:

a) performing a proliferation assay with cells expressing a mutant c-kit (for example in the transphosphorylase domain), which mutant is a permanent activated c-kit, with a plurality of test compounds to identify a subset of candidate compounds targeting activated c-kit, each having an IC50<10 μM, by measuring the extent of cell death,

b) performing a proliferation assay with cells expressing c-kit wild said subset of candidate compounds identified in step (a), said cells being IL-3 dependent cells cultured in presence of IL-3, to identify a subset of candidate compounds targeting specifically c-kit,

c) performing a proliferation assay with cells expressing c-kit, with the subset of compounds identified in step b) and selecting a subset of candidate compounds targeting c-kit wild, each having an IC50<10 μM, preferably an IC50<1 μM, by measuring the extent of cell death.

Here, the extent of cell death can be measured by 3H thymidine incorporation, the trypan blue exclusion method or flow cytometry with propidium iodide. These are common techniques routinely practiced in the art.

Therefore, the invention embraces the use of the compounds defined above to manufacture a medicament for treating bacterial infections in mammalian, especially in human. Such medicament is particularly useful for the treatment of recurrent bacterial infections, more particularly resurging infections after asymptomatic periods such as bacterial cystitis and respiratory tract infections. Preferably, the invention contemplates the use of the compounds defined above to manufacture a medicament for treating FimH expressing bacteria infections such as Gram-negative enterobacteria which include but are not limited to E. coli, Klebsiella pneumoniae, Serratia marcescens, Citrobactor freudii and Salmonella typhimurium.

The pharmaceutical compositions utilized in this invention may be administered by any number of routes including, but not limited to, oral, intravenous, intramuscular, intra-arterial, intramedullary, intrathecal, intraventricular, transdermal, subcutaneous, intraperitoneal, intranasal, enteral, topical, sublingual, or rectal means.

In addition to the active ingredients, these pharmaceutical compositions may contain suitable pharmaceutically-acceptable carriers comprising excipients and auxiliaries which facilitate processing of the active compounds into preparations which can be used pharmaceutically. Further details on techniques for formulation and administration may be found in the latest edition of Remington's Pharmaceutical Sciences (Maack Publishing Co., Easton, Pa.).

Pharmaceutical compositions for oral administration can be formulated using pharmaceutically acceptable carriers well known in the art in dosages suitable for oral administration. Such carriers enable the pharmaceutical compositions to be formulated as tablets, pills, dragees, capsules, liquids, gels, syrups, slurries, suspensions, and the like, for ingestion by the patient.

Pharmaceutical preparations for oral use can be obtained through combination of active compounds with solid excipient. Suitable excipients are carbohydrate or protein fillers, such as sugars, including lactose, sucrose, mannitol, or sorbitol; starch from corn, wheat, rice, potato, or other plants; cellulose, such as methyl cellulose, hydroxypropylmethyl-cellulose, or sodium carboxymethylcellulose; gums including arabic and tragacanth; and proteins such as gelatin and collagen. If desired, disintegrating or solubilizing agents may be added, such as the cross-linked polyvinyl pyrrolidone, agar, alginic acid, or a salt thereof, such as sodium alginate.

Dragee cores may be used in conjunction with suitable coatings, such as concentrated sugar solutions, which may also contain gum arabic, talc, polyvinylpyrrolidone, carbopol gel, polyethylene glycol, and/or titanium dioxide, lacquer solutions, and suitable organic solvents or solvent mixtures. Dyestuffs or pigments may be added to the tablets or dragee coatings for product identification or to characterize the quantity of active compound, i.e., dosage.

Pharmaceutical preparations which can be used orally include capsules made of gelatin, as well as soft, sealed capsules made of gelatin and a coating, such as glycerol or sorbitol. Push-fit capsules can contain active ingredients mixed with a filler or binders, such as lactose or starches, lubricants, such as talc or magnesium stearate, and, optionally, stabilizers. In soft capsules, the active compounds may be dissolved or suspended in suitable liquids, such as fatty oils, liquid, or liquid polyethylene glycol with or without stabilizers.

Pharmaceutical formulations suitable for parenteral administration may be formulated in aqueous solutions, preferably in physiologically compatible buffers such as Hanks' solution, Ringer's solution, or physiologically buffered saline. Aqueous injection suspensions may contain substances which increase the viscosity of the suspension, such as sodium carboxymethyl cellulose, sorbitol, or dextran. Additionally, suspensions of the active compounds may be prepared as appropriate oily injection suspensions. Suitable lipophilic solvents or vehicles include fatty oils such as sesame oil, or synthetic fatty acid esters, such as ethyl oleate or triglycerides, or liposomes. Non-lipid polycationic amino polymers may also be used for delivery. Optionally, the suspension may also contain suitable stabilizers or agents which increase the solubility of the compounds to allow for the preparation of highly concentrated solutions.

The pharmaceutical composition may be provided as a salt and can be formed with many acids, including but not limited to, hydrochloric, sulfuric, acetic, lactic, tartaric, malic, and succine, acids, etc. Salts tend to be more soluble in aqueous or other protonic solvents than are the corresponding free base forms. In other cases, the preferred preparation may be a lyophilized powder which may contain any or all of the following: 1-50 mM histidine, 0.1%-2% sucrose, and 2-7% mannitol, at a pH range of 4.5 to 5.5, that is combined with buffer prior to use.

Pharmaceutical compositions suitable for use in the invention include compositions wherein c-kit inhibitors are contained in an effective amount to achieve the intended purpose. The determination of an effective dose is well within the capability of those skilled in the art. A therapeutically effective dose refers to that amount of active ingredient, which ameliorates the symptoms or condition. Therapeutic efficacy and toxicity may be determined by standard pharmaceutical procedures in cell cultures or experimental animals, e.g., ED50 (the dose therapeutically effective in 50% of the population) and LD50 (the dose lethal to 50% of the population). The dose ratio of toxic to therapeutic effects is the therapeutic index, and it can be expressed as the ratio, LD50/ED50. Pharmaceutical compositions which exhibit large therapeutic indices are preferred. As mentioned above, a tyrosine kinase inhibitor and more particularly a c-kit inhibitor according to the invention is unable to promote death of IL-3 dependent cells cultured in presence of IL-3.

In another embodiment, the invention is aimed at a product comprising a tyrosine kinase inhibitor, more particularly a c-kit inhibitor, and at least one antibiotic selected bacitracin, the cephalosporins, the penicillins, the aminoglycosides, the tetracyclines, the streptomycins and the macrolide antibiotics such as erythromycin; the fluoroquinolones, actinomycin, the sulfonamides and trimethoprim for a separate, sequential or simultaneous use for treating recurrent bacterial infections, resurging infections after asymptomatic periods such as bacterial cystitis and respiratory tract infections.

This product is particularly useful for treating FimH expressing bacteria infections such as Gram-negative enterobacteria including E. coli, Klebsiella pneumoniae, Serratia marcescens, Citrobactor freudii and Salmonella typhimurium. Preferably, said inhibitor is unable to promote death of IL-3 dependent cells cultured in presence of IL-3 and the product further comprises an acceptable pharmaceutical carrier suitable for oral administration.

1. A method for treating bacterial infections comprising administering a tyrosine kinase inhibitor to a mammalian in need of such treatment, wherein said inhibitor is unable to promote death of IL-3 dependent cells cultured in presence of IL-3.

2. A method according to claim 1, wherein said tyrosine kinase inhibitor is a non-toxic, selective and potent c-kit inhibitor.

3. A method according to claim 2, wherein said inhibitor is selected from the group consisting of indolinones, pyrimidine derivatives, pyrrolopyrimidine derivatives, quinazoline derivatives, quinoxaline derivatives, pyrazoles derivatives, bis monocyclic, bicyclic or heterocyclic aryl compounds, vinylene-azaindole derivatives and pyridyl-quinolones derivatives, styryl compounds, styryl-substituted pyridyl compounds, , seleoindoles, selenides, tricyclic polyhydroxylic compounds and benzylphosphonic acid compounds.

4. A method for treating bacterial infections comprising administering a non toxic, potent and selective c-kit inhibitor to a mammalian in need of such treatment, selected from the group consisting of:
pyrimidine derivatives, more particularly N-phenyl-2-pyrimidine-amine derivatives,
indolinone derivatives, more particularly pyrrol-substituted indolinones,
monocyclic, bicyclic aryl and heteroaryl compounds,
and quinazoline derivatives.

5. A method according to claim 2, wherein said inhibitor is selected from the group consisting of N-phenyl-2-pyrimidine-amine derivatives having the formula II: 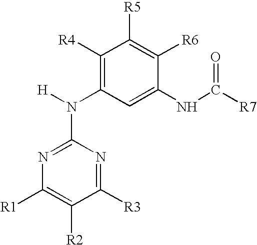
Wherein R1, R2 and R3 are independently chosen from H, F, Cl, Br, I, a C1-C5 alkyl or a cyclic or heterocyclic group, especially a pyridyl group;
R4, R5 and R6 are independently chosen from H, F, Cl, Br, I, a C1-C5 alkyl, especially a methyl group;
and R7 is a phenyl group bearing at least one substituent, which in turn possesses at least one basic site, such as an amino function, preferably the following group:

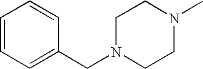

6. A method according to claim 5, wherein said inhibitor is the 4-(4-méhylpipérazine-1-ylméthyl)-N-[4-méthyl-3-(4-pyridine-3-yl)pyrimidine-2 ylamino)phényl]-benzamide.

7. A method according to one of claims 2 to 6, wherein said c-kit inhibitor is unable to promote death of IL-3 dependent cells cultured in presence of IL-3.

8. A method according to one of claims 2 to 7, wherein said inhibitor is an inhibitor of activated c-kit selected from a constitutively activated-mutant c-kit and/or SCF-activated c-kit.

9. A method according to claim 8, wherein the activated-mutant c-kit has at least one mutation selected from mutations proximal to Y823, more particularly between amino acids 800 to 850 of SEQ ID No1 involved in c-kit autophosphorylation, notably the D816V, D816Y, D816F and D820G mutants, and a deletion in the juxtamembrane domain of c-kit, preferably between codon 573 and 579.

10. A method for treating bacterial infections comprising administering to a mammalian in need of such treatment a compound that is a selective, potent and non toxic inhibitor of activated c-kit obtainable by a screening method which comprises:
a) bringing into contact (i) activated c-kit and (ii) at least one compound to be tested; under conditions allowing the components (i) and (ii) to form a complex,
b) selecting compounds that inhibit activated c-kit,
c) testing and selecting a subset of compounds identified in step b), which are unable to promote death of IL-3 dependent cells cultured in presence of IL-3.

11. A method according to claim 10, wherein the screening method further comprises the step consisting of testing and selecting a subset of compounds identified in step b) that are inhibitors of mutant activated c-kit, which are also capable of inhibiting SCF-activated c-kit wild.

12. A method according to claim 10, wherein activated c-kit is SCF-activated c-kit wild.

13. A method according to one of claims 10 to 12, wherein putative inhibitors are tested at a concentration above 10 μM in step a).

14. A method according to one of claims 10 to 13, wherein IL-3 is present in the culture media of IL-3 dependent cells at a concentration comprised between between 0.5 and 10 ng/ml, preferably between 1 to 5 ng/ml.

15. A method according to one of claims 10 to 14, wherein the extent to which component (ii) inhibits activated c-kit can be measured in vitro or in vivo.

16. A method according to one of claims 10 to 15, wherein the screening method further comprises the step consisting of testing and selecting in vitro or in vivo compounds capable of inhibiting c-kit wild at concentration below 1 μM.

17. A method according to claim 16, wherein the test is performed using cells lines selected from the group consisting of mast cells, transfected mast cells, BaF3, and IC-2.

18. A method according to claim 16, wherein the test includes the determination of the amount of c-kit phosphorylation.

19. A method for treating bacterial infections according to one of claims 10 to 18, wherein the screening comprises:
a) performing a proliferation assay with cells expressing a mutant c-kit (for example in the transphosphorylase domain), which mutant is a permanent activated c-kit, with a plurality of test compounds to identify a subset of candidate compounds targeting activated c-kit, each having an IC50<10 μM, by measuring the extent of cell death,
b) performing a proliferation assay with cells expressing c-kit wild said subset of candidate compounds identified in step (a), said cells being IL-3 dependent cells cultured in presence of IL-3, to identify a subset of candidate compounds targeting specifically c-kit,
c) performing a proliferation assay with cells expressing c-kit, with the subset of compounds identified in step b) and selecting a subset of candidate compounds targeting c-kit wild, each having an IC50<10 μM, preferably an IC50<1 μM, by measuring the extent of cell death.

20. A method according to one of claims 1 to 19 for treating recurrent bacterial infections, more particularly resurging infections after an asymptomatic period.

21. A method according to one of claims 1 to 19 for treating bacterial infections, wherein bacteria are FimH expressing bacteria such as Gram-negative enterobacteria such as E. coli, Klebsiella pneumoniae, Serratia marcescens, Citrobactor freudii and Salmonella typhimurium.

22. A method according to one of claims 1 to 19 for treating urinary tract infections such as bacterial cystitis and respiratory tract infections.

23. A method according to one of claims 20 to 22, wherein the inhibitor is administered orally.

24. Use of a tyrosine kinase inhibitor, more particularly a c-kit inhibitor, to manufacture a medicament for treating bacterial infections in mammalian, especially in human, preferably for the treatment of recurrent bacterial infections, resurging infections after asymptomatic periods such as bacterial cystitis and respiratory tract infections.

25. Use of a tyrosine kinase inhibitor, more particularly a c-kit inhibitor, to manufacture a medicament for treating FimH expressing bacteria infections such as Gram-negative enterobacteria including E. coli, Klebsiella pneumoniae, Serratia marcescens, Citrobactor freudii and Salmonella typhimurium.

26. A product comprising a tyrosine kinase inhibitor, more particularly a c-kit inhibitor, and at least one antibiotic selected from bacitracin, the cephalosporins, the penicillins, the aminoglycosides, the tetracyclines, the streptomycins and the macrolide antibiotics such as erythromycin; the fluoroquinolones, actinomycin, the sulfonamides and trimethoprim for a separate, sequential or simultaneous use for treating recurrent bacterial infections, resurging infections after asymptomatic periods such as bacterial cystitis and respiratory tract infections.

27. A product according to claim 26 for treating FimH expressing bacteria infections such as Gram-negative enterobacteria including E. coli, Klebsiella pneumoniae, Serratia marcescens, Citrobactor freudii and Salmonella typhimurium.

28. A product according to one of claims 26 and 27, wherein said c-kit inhibitor is unable to promote death of IL-3 dependent cells cultured in presence of IL-3.

29. A product according to one of claims 26 and 27, further comprising an acceptable pharmaceutical carrier suitable for oral administration.
